(19) United States
(12) Patent Application Publication
(10) Pub. No.: US 2022/0087593 A1
(43) Pub. Date: Mar. 24, 2022

(54) DEVICE FOR MEASURING A CONGESTION OF THE DIGESTIVE TRACT

(71) Applicants: SENTINHEALTH, Paris (FR); UNIVERSITE GRENOBLE ALPES, Saint Martin D'heres (FR)

(72) Inventors: Cindy DOPIERALA, Paris (FR); Adrien POIZAT, Paris (FR); Thibault PARMENTIER, Paris (FR); Pierre-Yves GUMERY, Grenoble (FR)

(21) Appl. No.: 17/423,932

(22) PCT Filed: Jan. 24, 2020
(86) PCT No.: PCT/FR2020/050114

§ 371 Date: Jul. 19, 2021

(30) Foreign Application Priority Data
Jan. 24, 2019 (FR) 1900629

(51) Int. Cl. A61B 5/00; A61B 5/053; A61B 5/279

(52) U.S. Cl. CPC A61B 5/42; A61B 5/053; A61B 2562/0219; A61B 5/6873; A61B 5/279

(57) ABSTRACT

A device (1) for measuring congestion of the digestive tract comprises at least one housing (2), a current generator (30) and a means (31) for measuring a difference in potential, said generator and means being accommodated in said housing, and a set of electrodes (3) comprising at least two electrodes connected electrically, independently of one another, to the current generator (30) and/or to the means (31) for measuring a difference in potential across the terminals of the electrodes, each electrode of the set (3) of electrodes being configured to transmit an electric current and/or to allow a difference in electrical potential to be measured.

The set (3) of electrodes is configured to generate at least one electric current flow loop (13) flowing at least through a tissue of the gastrointestinal tract (14) of the user and to allow a difference in electrical potential in relation to the tissue of the gastrointestinal tract to be measured, the device (1) for measuring the bio-impedance of the digestive tract further comprising a calculating module (5) configured to receive the measured difference in electrical potential and to calculate a value of the bio-impedance of the digestive tract according to this measurement in relation to the tissue of the digestive tract.

The present invention relates to the field of measuring devices applied to bodily components, more particularly devices capable of measuring the bio-impedance of a human body in the digestive tract. The term “tissue” used below will denote all the tissues of the digestive tract concerned by the impedance measurement, and more precisely the tissues of the gastrointestinal tract.

In a known manner, pathologies such as heart failure can cause an accumulation of fluid in the lungs in humans. This aqueous accumulation, also called edema, prevents correct breathing: the water that becomes lodged in the pulmonary tissues at the extracellular level constitutes an obstacle to the gaseous exchanges of oxygen and carbon dioxide which take place there.

Cardiac decompensation can thus lead to cardiogenic edema in the chest, especially in the lung tissue. Breathing discomfort and shortness of breath are among the symptoms, induced by a compensation of the lungs increasing their work of breathing, and in particular causing chest pain. These symptoms worsen to major respiratory distress if the accumulation of fluid is not detected upstream. Pulmonary edema is medically considered to be a vital emergency that must be treated at the first signs; the treatments are heavier the later the diagnosis is made, as the tissues are less engorged at an initial stage.

Pulmonary edema is a late stage of the disease, the treatment of which is based, among other things, on the use of diuretic drugs; the effectiveness of these drugs at this stage of the disease is insufficient by the oral route (even at high doses), requiring hospitalization and intravenous diuretic treatment. The explanation for the decreased effectiveness of these oral medications lies in the presence of water (edema) in the gastrointestinal wall, preventing their absorption. It is even likely, due to the seriousness, that the pulmonary edema is posterior to the edema of the gastric and intestinal wall. Thus, measuring the presence of water in the digestive tract could be an early sign of the subsequent stage, namely the presence of water in the lungs, especially since 35% of patients with heart failure have an increased digestive permeability (Sandek A, Bauditz J, Swidsinski A, Buhner S, Weber-Eibel J, von Haehling S et al. Altered Intestinal Function in Patients With Chronic Heart Failure. JACC October 2007. Vol. 50, No. 16, 2007, 2007:1561-9)

Any patient at risk for cardiac pathology must therefore be vigilant because of the possible appearance of pulmonary edema. At-risk patients are generally monitored through regular medical examinations, such as auscultation by the practitioner, chest x-ray, blood test and/or electrocardiogram in order to identify cardiac disorders. The patient is forced to engage in close preventative monitoring of his lifestyle and to treat his cardiac pathology to avoid underlying complications of pulmonary edema.

However, medical monitoring remains restrictive for the patient, since he is dependent on the medical setting and the practitioner to assess his pulmonary and cardiac condition. Another drawback lies in the regularity of the follow-up, the patient having to submit to the medical setting frequently to prevent worsened complications. In addition, long-term multi-day monitoring cannot reasonably be done for all patients, especially those who maintain good autonomy.

The aim of the present invention is therefore to address the drawbacks described above by designing a bio-impedance measuring device used to robustly detect changes in water content, or congestion, in gastric tissue in order to detect the development of visceral edema, which is simple to use and can be used multiple times for the indirect and early monitoring of a patient at risk for pulmonary edema.

The invention relates to a device for measuring congestion of the digestive tract of a user, the measuring device comprising at least one housing, a current generator and a means for measuring a difference in potential, said generator and means each being accommodated in said housing, and a set of electrodes comprising at least two electrodes connected electrically, independently of one another, to the current generator and/or to the means for measuring a difference in potential across the terminals of the electrodes, each electrode of the set of electrodes being configured to transmit an electric current and/or to allow a difference in electrical potential to be measured. The set of electrodes is configured to generate at least one electric current flow loop flowing at least through a tissue of the gastric or intestinal tract of the user and to allow a difference in electrical potential in relation to the tissue of the gastric or intestinal tract to be measured, and the measuring device further comprises a calculating module configured to receive the measured difference in electrical potential and to calculate a value of the bio-impedance of the digestive tract according to this measurement in relation to the tissue of the gastric or intestinal tract. Such a device for measuring congestion of the digestive tract thus makes it possible in particular to measure a bio-impedance of the tissues of the digestive tract.

An automated measurement of a variation in the volume of fluid in the gastric or intestinal tissue would make it possible to assess the appearance of edema with relative autonomy. Measuring bio-impedance is a measurement providing information in particular on a liquid composition of the body, such as the presence of edema. Known devices for measuring bio-impedance do not, however, make it possible to specifically and robustly assess the presence of thoracic edema. In addition, no known device makes it possible to assess the presence of edema of the digestive tract.

According to one feature of the invention, the measuring device comprises distinct means allowing a mechanical measurement of a structural characteristic of the gastric wall.

The measuring device according to the invention thus implements, within the framework of a multimodal approach, means for measuring an electrical impedance of the gastric tissue and distinct means allowing a mechanical measurement of a structural or morphological characteristic of the gastric wall.

The measuring device can then be defined as multimodal in that it allows the measurement and taking into account of electrical bio-impedance signals on the one hand and of mechanical signals on the other hand. Cross-analysis of these data, and for example when each of these data exceeds a threshold value, makes it possible to reliably determine whether the gastric tissue is congested, i.e., whether it is filled with fluid, such a determination subsequently allowing a medical professional to diagnose possible heart failure.

According to one feature of the invention, the means allowing a mechanical measurement of a structural or morphological characteristic of the gastric wall consist of an accelerometer. Such a sensor is in particular configured to detect vibrations and to quantify, by at least one defined quantity, these vibrations at the gastric wall.

It is particularly advantageous to link the evolution of the characteristics of the seismocardiography waves in response to a cardiac shock, by analysis of the accelerometric signal collected by the accelerometer, with the different levels of electrical impedance of the gastric tissue. Analyzing the characteristics of the accelerometric signal, for example the energy or the frequency bandwidth of the vibrations of the gastric wall, makes it possible to highlight indications of structural and morphological modifications of the gastric wall. Combining this information with the analysis of the impedance levels makes it possible to deduce that the detected structural and morphological change is due to an increased presence of fluid in the gastric wall.

A learning base using machine learning methodologies can be used to detect characteristic variations related to the onset of visceral edema.

According to one feature of the invention, the means allowing a mechanical measurement of a structural or morphological characteristic of the gastric wall, in particular the accelerometer, are integrated into the housing of the previously mentioned measuring device.

Advantageously, the means allowing a mechanical measurement of a structural or morphological characteristic of the gastric wall, in particular the accelerometer, are thus arranged near the electrodes allowing the measurement of an accelerometric signal in an area of the gastric tissue corresponding to the area in which the bio-impedance measurement was carried out. In this context, it is also easier to collect the measured data in a single calculating module, configured to process the measured data and in particular to compare them with threshold values in order to detect, if necessary, the exceeding of an alert level that is likely to trigger a subsequent medical diagnostic process.

This mechanical proximity, due to the integration of the sensors and electrodes into the same housing, also makes it possible to optimize the synchronization of the measurements.

As will be mentioned below, the measurements of bio-impedance and of the mechanical signal, here accelerometric, are carried out simultaneously in order to have a reliable basis of comparison, and the synchronization of the launching of these two measurements is facilitated by a short path of the control instructions between the calculating module, for example, and the sensors and electrodes.

The housing and the set of electrodes are configured to be internalized, that is to say, implanted in the user. They are configured so that at least the housing and, where appropriate all or part of the electrodes, are in direct contact with the tissues of the user, and more particularly in prolonged contact with the tissues of the user. In this context, the housing and the set of electrodes can be made from materials which are inert to the body. For example, the housing and the set of electrodes can be made from biocompatible materials or comprise a biocompatible coating. Regardless of the materials used, the set of electrodes retains its conducting transmitting and/or receiving properties.

The calculating module is configured to process the measurement data for the difference in electrical potential, and in particular to process the evolution of these measured data with respect to predefined thresholds, and to deduce therefrom a bio-impedance value of the gastric tract, from which it is possible to extract information relating to the user's condition. It comprises at least communication means enabling it to receive the difference in electrical potential measured across the terminals of the electrodes supplied by the current generator. The device used to measure this difference in electrical potential, namely a voltmeter, and the calculating module are wired or communicate by waves, depending on the embodiment. The calculating module can be embedded in the housing or else be offset at a distance from the housing, without departing from the context of the invention, provided that the housing incorporates at least one current generator and one measuring device capable of being electrically connected, independently of each other, to the electrodes, which may or may not be offset from the housing.

Whether it is on-board or offset from the housing, the calculating module can, in one embodiment, be configured to be internalized, that is to say, implanted in the user. It then comprises means of communication allowing it to transmit the calculated bio-impedance value of the digestive tract, or the information relating to a condition of the user. For example, the calculating module comprises transmitters. In another embodiment, the calculating module is externalized.

The set of electrodes with at least two electrodes, whether these are integrated into the housing or offset outside the housing, is connected to the current generator and to the means for measuring a difference in potential, each electrode of the set, independently of one another, being electrically connected to these components, while being positioned at least in contact with the gastric or intestinal tissue through which it is desired to pass the current flow loop. For example, each of the electrodes of the set of electrodes comprises at least one interface surface intended to be in contact with gastric or intestinal tissue. Advantageously, the set of electrodes is configured to be placed in the gastric or intestinal tissue.

The electric current caused to flow through the user's body via the electrodes is a low-intensity electric current. “Low intensity” in particular means a current of intensity less than one milliampere. More particularly, by way of example, the electric current can be a sinusoidal alternating current, with an intensity on the order of 50 to 700 microamperes for a frequency on the order of 5 kHz to 1 MHz, the measurement possibly being a multi-frequency measurement.

The measurement of the difference in electrical potential is carried out across the terminals of the electrodes, for a current of constant intensity, of an impedance value, according to Ohm's law. The difference in electrical potential resulting from the passage of current from one electrode to another, in one direction or the other, it being understood that the electrodes can be both injecting and receiving, is modified according to the resistance of the biological tissues encountered in the gastric or intestinal area passed through. The measurement across the terminals of the electrodes arranged on the current path makes it possible to quantify the variation of this electrical potential and therefore the variation in bio-impedance for a current of constant intensity.

The measurement of electrical quantities of flowing current which is carried out by the device according to the invention meets several requirements. A local measurement should be carried out in order to be able to perform a sufficiently specific measurement of the bio-impedance in the gastric or intestinal tissue, and for this local measurement to be targeted in the submucosa and/or where appropriate in the mucosa, in order to avoid a possible electric shunt due to the contents of the gastric or intestinal lumen. This measurement should also be carried out over a sufficiently large distance to have a reliable overall measurement that does not take into account the heterogeneity of the tissues present in these different walls. And in this context, different features of the invention as they will be described in more detail below aim to provide a device in which a plurality of electrodes are arranged redundantly one behind the other, electrically connected independently of each other to the current generator, making step-by-step measurements possible without the current flow loops extending detrimentally into the stomach cavity.

According to the invention, an accelerometric measurement is carried out at time t. The values collected by the accelerometer are sent to the calculating module to extract the accelerometric signature of the gastric wall in response to the cardiac shock. As has been mentioned, the calculating module can be invasive and arranged in the housing of the measuring device or can be externalized without departing from the context of the invention, since the mechanical and electrical means are located in the same housing. At the same time, an electrical impedance value of the gastric tissue is measured. The accelerometric signature of the gastric wall and the electrical impedance values over time are analyzed in particular by comparing them with threshold values. In the event that the evolution of these two measurements, electrical and mechanical, is consistent, and that the collected values each exceed a corresponding threshold value defined beforehand, information is sent to a database and/or an external data processing means, it being understood that this information is to be considered as a possible visceral edema to be diagnosed later.

The inventors have in fact considered that in the case of fluid retention of the gastric wall, on the one hand the electrical impedance of the tissue will vary, the resistance decreasing due to the presence of water which facilitates the transfer of the current from one electrode of the housing to the other, and on the other hand the gastric wall also undergoes structural modifications due to the presence of water, including a thickening of the wall, which causes a modification of the accelerometric signature, the waves caused by the cardiac shock then not being transmitted in the same way.

Taking into account the electrical and mechanical signals acquired simultaneously by virtue of the measuring device according to the invention thus makes it possible to provide reliable information on the existence of water retention.

According to one aspect of the invention, the measuring device is sized to be implanted in the tissue of the gastrointestinal tract by the endo-luminal route or by abdominal surgery. To be implanted at the tissue of the gastrointestinal tract via the endo-luminal route, the measuring device is sized to be inserted through the user's oral cavity and digestive tract. For example, the measuring device is sized to be inserted into or carried by an endo-luminal probe, such as an endoscope. The endo-luminal implantation can be coupled with a surgical act intended to finalize the implantation of the measuring device. Alternatively, the measuring device is intended to be implanted by a surgical act performed directly in the abdomen.

According to one aspect of the invention, the set of electrodes comprises two electrodes, namely a first electrode arranged at a first longitudinal end of the housing and a second electrode arranged at an opposite second longitudinal end of the housing. Arranging the set of electrodes at the ends of the housing makes it possible to measure the difference in electrical potential locally, that is to say, over the distance of a few centimeters presented by the housing. The flow loop thus passes from the first electrode to the second electrode, passing through the gastric or intestinal tissue near the housing, between the first longitudinal end and the second longitudinal end of the housing. It will be understood that this measurement, which is simple because it does not involve electrodes or electrical connection wires outside the housing, involves a very local measurement, which can vary greatly depending on the implantation area of the housing as a function of the heterogeneity of the tissues.

The first electrode comprises a first interface surface configured to contact a first area of the gastrointestinal tract. The second electrode comprises a second interface surface configured to contact a second area of the gastrointestinal tract, distinct from the first. For example, so that the first electrode/first gastric or intestinal area contact is ensured just like the second electrode/second gastric or intestinal area contact, the first interface surface is flush with the first longitudinal end of the housing and the second interface surface is flush with the second longitudinal end of the housing.

The interface surface of each electrode is extended by a certain surface area. For example, without limiting the invention, the interface surface of each electrode may be between 20 and 40 mm². In another example, the interface surface of each electrode may correspond to 15% of the total surface of the housing, +/−10%.

The first electrode is, in one embodiment, extended over the entire first longitudinal end of the housing. In this same embodiment, the second electrode can also be extended over the entire second longitudinal end of the housing. In an alternative embodiment, the first electrode occupies part of the first longitudinal end of the housing. In this same embodiment, the second electrode can also occupy part of the second longitudinal end of the housing. For example, the first electrode and the second electrode are in the form of a sticker placed on the surface of the housing respectively at the first longitudinal end and the second longitudinal end. Advantageously, the first interface surface and the second interface surface are oriented in the same direction; the first electrode and the second electrode are then arranged on the same side on the housing, the normals to each interface surface being parallel.

According to one aspect of the invention, the set of electrodes comprises a plurality of electrodes among which at least one electrode is offset outside the housing, and in which an offset electrode support external to the housing is configured to connect one of the terminals of the current generator and/or of the means for measuring a difference in potential to said offset electrode, the offset electrode support comprising an insulating peripheral sheath extending between the housing and said offset electrode

According to one aspect of the invention, the set of electrodes comprises a plurality of electrodes, arranged geometrically one after the other moving away from the housing, where appropriate while being substantially aligned.

In this configuration, the greatest possible distance between two electrodes of the set of electrodes is increased compared to a configuration where the electrodes are accommodated in the housing. This makes it possible to have good coverage of the bio-impedance measurement field on the gastric or intestinal tissue, while the housing remains relatively small so as to minimize the invasive impact of the housing and of the offset electrode support.

The offset electrode support is located outside the housing. It is electrically insulated by the peripheral sheath which surrounds it, and connects at least one offset electrode to the current generator and/or to the means for measuring a difference in electrical potential. In order for the inter-electrode distances to be maintained when the measuring device according to the invention is installed, the offset electrode support may have a certain rigidity. For example, it is the insulating peripheral sheath which makes it possible to ensure this rigidity. Advantageously, the insulating peripheral sheath can be semi-rigid so as not to hamper the user.

In the foregoing and what will follow, the term “electrodes” is used to refer generally to a single electrode or to a set of electrodes arranged side by side in a defined area. The measuring device according to the invention can be implemented equally well with a two-electrode bio-impedance measurement, which is then understood as a current flow between a transmitter/receiver electrode and another transmitter/receiver electrode which are remote from one another, or with a bio-impedance measurement with four electrodes, which is then understood by a current flow between a set of transmitting electrodes and a set of receiving electrodes, these sets being at a distance from one another.

In the latter case in particular, a set of electrodes can be configured to inject current and form a flow loop and moreover to receive current from another flow loop when separate local measurements are implemented simultaneously or alternatively.

According to one aspect of the invention, all the electrodes of the set of electrodes are offset outside the housing, the insulating peripheral sheath accommodating, in a manner electrically insulated from one another, a plurality of electrically conductive cords configured to connect the offset electrodes to the current generator and/or to the means for measuring a difference in potential, independently of each other. It is possible to identify among the electrodes, arranged geometrically one after the other in the direction moving away from the housing, end electrodes with a first end electrode closest to the housing and a second end electrode furthest from the housing, and one or more intermediate electrodes. It should be noted that the term “intermediate” then refers to the geometric position of the electrode and not to its technical characteristics. The intermediate electrode(s) are offset from the housing.

According to one aspect of the invention, the set of electrodes forms a one-dimensional matrix extending from the housing to the electrode furthest from the housing, passing through each of the intermediate electrodes. The plurality of electrodes of the set of electrodes thus formed makes it possible to carry out several local measurements of the difference in electrical potential. Each local measurement of electrical potential corresponds to a local flow loop, corresponding to the shortest flow loop produced by the electrodes of the set of electrodes, step by step. This redundancy in the positioning of the electrodes, electrically connected to the current generator independently of each other, makes it possible to carry out measurements step by step and to extend the distance over which the measurement is carried out while ensuring that there is little to no penetration of the gastrointestinal lumen by the flow loops, inside which gastrointestinal lumen the conductivity of the gastric or intestinal luminal contents could distort the analysis of the measurement of the difference in electrical potential across the electrodes.

It is understood that a measurement at several successive points makes it possible to extend the scope of the measurement and therefore to propose a reliable overall measurement, not dependent on the heterogeneity of the tissues. By way of example, four intermediate electrodes can be provided arranged between the housing and the end electrode furthest from the housing, without this being limiting. It will be understood that it will be possible to propose, without departing from the context of the invention, a different number of electrodes as long as the closure of a current flow loop is generated step by step, as has been mentioned above.

According to one aspect of the invention, an equal distance can separate each electrode from its immediately adjacent electrode. In other words, the same inter-electrode distance separates the electrodes of the set of electrodes, two by two.

According to one aspect of the invention, each electrode offset from the housing comprises an interface surface with the gastric or intestinal tissue. The device can be implanted so that the interface surfaces of these electrodes are oriented in the same direction and the same sense, the direction and the sense of the interface surface being defined with respect to a normal to the plane in which the interface surface extends. The device can be implanted so that the interface surface of the electrodes is in contact with gastric or intestinal tissue, so as to ensure that the current flow loop passes as much as possible through gastric or intestinal tissue. Advantageously, the entire flow loop passes through gastric or intestinal tissue. Thus, the electrical potential measurement carried out, revealing the bio-impedance state of the digestive tract, is specific to the tissue and not to the adjacent environments, such as the cavity delimited by the gastric or intestinal wall.

According to one aspect of the invention, the set of electrodes comprises at least one intermediate electrode extending outside the housing between the housing and said offset electrode forming the electrode furthest from the housing, at least one intermediate electrode being arranged at the end of a current conducting branch and insulated from the tissue of the digestive tract by an insulating sheath, said branch being deployable relative to the cord provided with the peripheral insulating sheath.

The electric current flows in the electrically conductive cord of the offset electrode support and in the current conducting branch to supply the electrodes of the device independently of one another and to make it possible to form each flow loop to be considered in order to determine the gastric bio-impedance value. The deployable conductive branch makes it possible to move the electrode that it carries away from the offset electrode support. When the offset electrode support is implanted, moving the electrodes away makes it possible to avoid the fibrous degeneration of the tissue that occurs around the offset electrode support following implantation of the housing of the device to which the cord is connected. The measurement of the difference in electrical potential carried out is then more robust. In the operating position, the current conducting branch extends substantially perpendicularly to the offset electrode support. In order to be kept in this position, the current conducting branch is advantageously rigid.

Before the installation of the measuring device according to the invention, the current conducting branch can be in the folded or retracted position. In the folded or retracted position, the conductive branch extends along the offset electrode support. Once the measuring device is in the operating position, the conductive branch is in the deployed position so as to move the electrode it carries away from the offset electrode support. The deployable nature of the current conducting branch is compatible with deployment within gastric or intestinal tissue. Thus, the deployment must be done despite the resistance that the current conducting branch and the electrode that it carries may encounter. To be deployable, the current conducting branch is for example associated with a deployment mechanism. For example, the deployment mechanism is a detent or spring deployment mechanism.

The current conducting branch is biocompatible, as is the deployment mechanism that it contains.

According to one feature of the invention, the set of electrodes associated with the housing comprises a plurality of electrodes electrically connected to the current generator and/or to the means for measuring a difference in potential, whether the electrodes are accommodated in the housing or offset outside the housing, and the measuring device comprises a set of switches arranged on the electrical connections independently of each electrode with the current generator and/or the means for measuring a difference in potential, the calculating module being configured to drive the opening and closing of each switch to determine which electrodes in the set are implemented to transmit and measure the current through the gastric or intestinal tissue. Implementing these controlled switches makes it possible to select which pair of electrodes is chosen to form the current flow loop to be considered. And it is then possible to program a sequence of opening and closing of the switches in order to carry out a succession of local measurements between two adjacent electrodes, if necessary immediately adjacent electrodes, in order to then average these local measurements to define an overall measurement.

As previously described, the measuring device can be configured to allow two-electrode measurement, or four-electrode measurement. In the measurement with two electrodes, each electrode is connected both to the current generator and to the device for measuring the difference in potential, so as to operate by injecting and receiving current passing through the gastric or intestinal tissue. And in the measurement with four electrodes, one pair of electrodes is connected across the terminals of the current generator and another pair of electrodes is connected across the terminals of the means for measuring a difference in potential, each electrode being able to operate respectively in current transmission or reception.

According to one aspect of the invention, the measuring device comprises at least one device for attachment to the gastric or intestinal tissue. The attachment device corresponds to any element enabling the measuring device to be secured to the gastric or intestinal tissue. Thus immobilized, the device for measuring the bio-impedance of the digestive tract makes it possible to obtain reliable measurements of the difference in potential, since there is no approximation of the actual position of the electrodes with respect to one another. The attachment device is for example provided with a clamp suitable for attaching the measuring device to the gastric or intestinal tissue.

According to one aspect of the invention, at least one end of the housing comprises a device for attaching to the gastric or intestinal tissue. In other words, the measuring device may comprise a device for attaching to gastric or intestinal tissue arranged at the first longitudinal end and/or a device for attaching to the second longitudinal end. Advantageously, the device for attaching to the gastric or intestinal tissue is placed on each end of the housing opposite the interface surface of the electrodes. Thus, the flow of current is not disturbed.

According to one aspect of the invention, the offset electrode support comprises all or part of the device for attaching to the gastric or intestinal tissue. The attachment device can thus be present at the offset electrode support. The orientation and the position of the electrodes carried by the offset electrode support, namely the third electrode and potentially the series of intermediate electrodes, are ensured, guaranteeing the reproducibility of the electrical potential measurements. The offset electrode support can associate an attachment device with each electrode.

According to one aspect of the invention, at least the current conducting branch comprises a device for attaching to the gastric or intestinal tissue. When the current conducting branch is able to be deployed, the attachment device is positioned so as not to hinder this deployment. The attachment device, or anchoring device, is attached to the gastric or intestinal tissue, after the deployment of the current conducting branch.

Thus, at least one end of the housing and/or the offset electrode support and/or the current conducting branch comprises the device for attaching to the gastric or intestinal tissue.

The invention also relates to a method for measuring congestion of the digestive tract, implementing the measuring device as described above, the method comprising a step of measuring the difference in electrical potential and a calculating step, the calculating step being implemented by a calculating module capable of receiving at least one measurement of the difference in electrical potential and of calculating a bio-impedance value of the digestive tract from the measurement of the difference in electrical potential.

The step for measuring the difference in electrical potential includes the injection of current through the electrodes capable of injecting current and the reception by the receiving electrodes of the current which has passed through the flow loop through the gastric or intestinal tissue.

The calculation step is implemented by the calculating module. The calculating module integrates the measurement of the difference in electrical potential or the measurements of the local difference in electrical potential, depending on the number of electrodes used in the set of electrodes. The calculating module determines a bio-impedance value of the digestive tract owing to the measurements of the difference in electrical potential carried out across the terminals of the electrodes.

According to one aspect of the invention, the measurement of the difference in electrical potential corresponds to the sum of a plurality of local measurements of the difference in electrical potential, each local measurement of the difference in electrical potential resulting from an electrical connection to at least one current generator of a pair of immediately adjacent electrodes of the electrode set of the measuring device. During the measurement step, the local measurements of differences in electrical potential are collected. Each local measurement of differences in electrical potential corresponds to the difference in electrical potential measured for the shortest possible flow loop, for the electrodes step by step.

Other features, details and advantages of the invention will emerge more clearly on reading the description given below by way of indication, in relation to the drawings, in which:

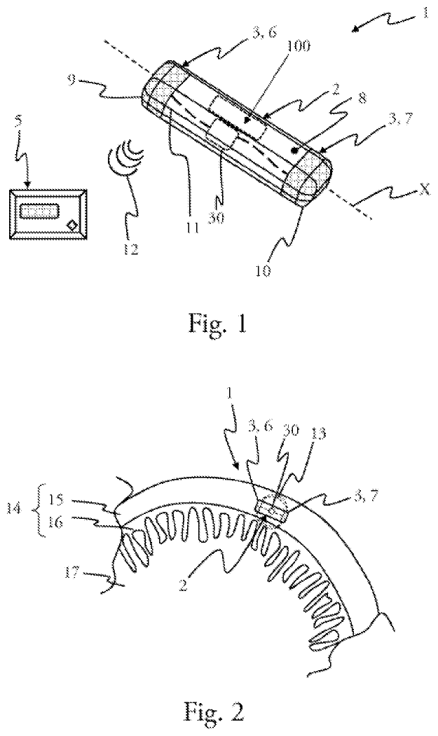

FIG. 1 is a general perspective view of a device for measuring congestion of the digestive tract according to the invention in a first embodiment,

FIG. 2 is a view of the implanted elements of the measuring device shown in FIG. 1 in an operating mode,

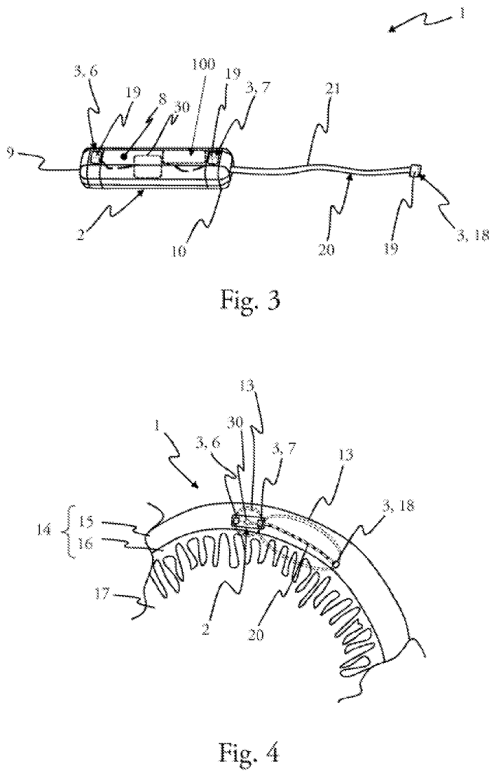

FIG. 3 is a general perspective view of the elements intended to be implanted of a measuring device according to the invention in a second embodiment,

FIG. 4 is a view of the implanted elements of the measuring device shown in FIG. 3 in an operating mode,

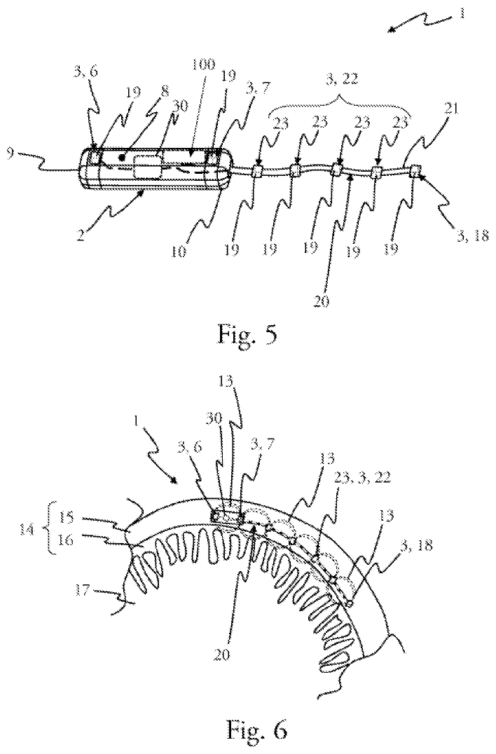

FIG. 5 is a general perspective view of the elements intended to be implanted of a measuring device according to the invention in a third embodiment,

FIG. 6 is a view of the implanted elements of the measuring device shown in FIG. 3 in an operating mode,

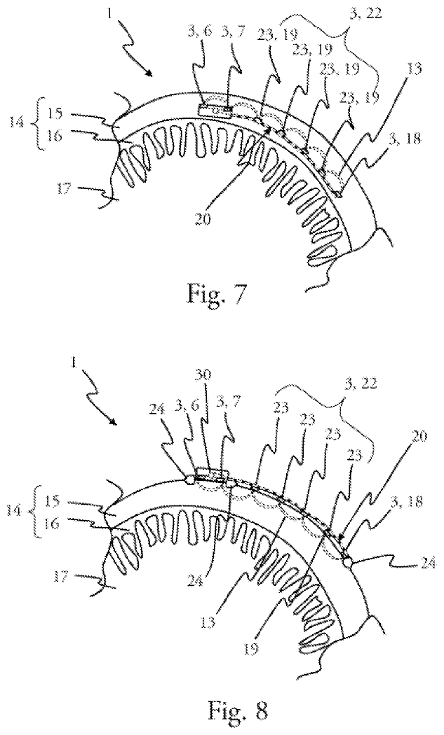

FIG. 7 and FIG. 8 are in situ views of the implanted elements of the measuring device according to the invention in a fourth and a fifth embodiment,

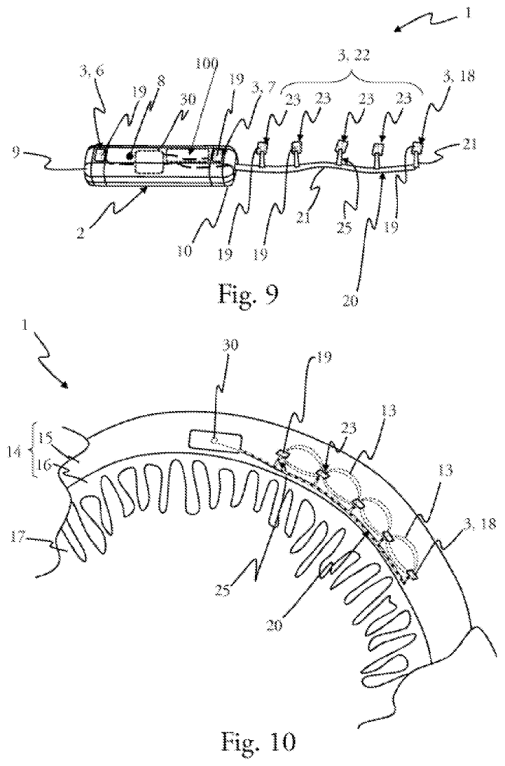

FIG. 9 is a general perspective view of the elements intended to be implanted of a measuring device according to the invention in a sixth embodiment,

FIG. 10 is a view of the implanted elements of the measuring device shown in FIG. 9 in an operating mode,

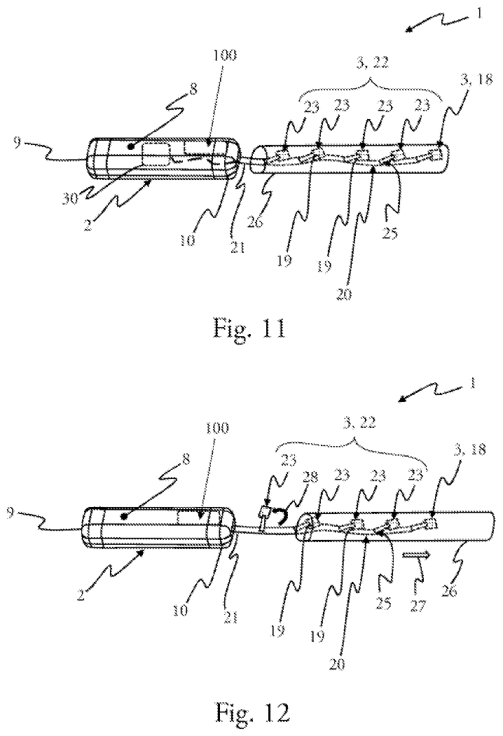

FIG. 11 and FIG. 12 describe a method of installing the elements intended to be implanted of the measuring device shown in FIG. 9,

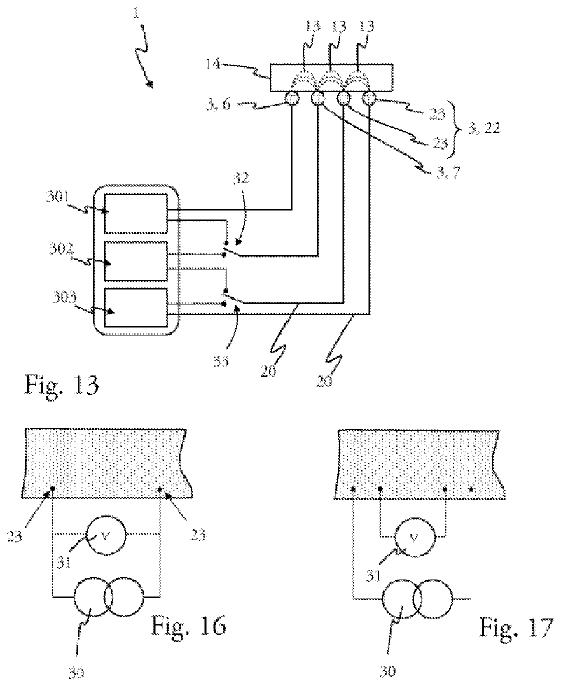

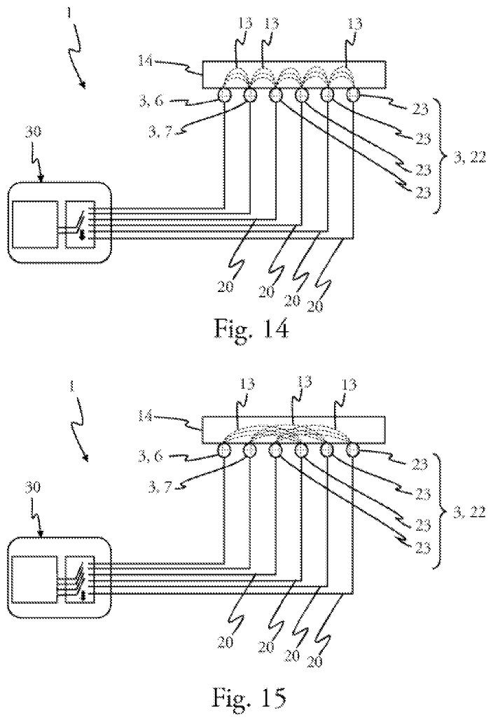

FIG. 13, FIG 14 and FIG. 15 illustrate appropriate electrical connection diagrams for the correct operation of the measuring device according to the invention, and

FIG. 16 and FIG. 17 respectively illustrate, in more detail, the operation of a so-called two-electrode and four-electrode measurement.

It should first of all be noted that the figures set out the invention in detail for its implementation, these figures of course being able to be used to better define the invention where appropriate.

In the remainder of the description, the names “longitudinal” or “lateral,” “above,” “below,” “in front,” “behind” refer to the orientation of the housing of the device for measuring congestion of the digestive tract according to the invention. The longitudinal direction corresponds to the main axis of the housing in which it extends, while the lateral orientations correspond to concurrent straight lines, that is to say, which intersect the longitudinal direction, in particular perpendicular to the longitudinal axis of the housing.

Referring firstly to FIG. 1, a device 1 is shown for measuring congestion of the digestive tract of a user. The measuring device 1 comprises at least one housing 2, a set 3 of electrodes connected to the housing 2 as well as a current generator 30 accommodated in the housing to which each electrode of the set 3 of electrodes is connected electrically and independently of one another. The measuring device 1 further comprises a means for measuring a difference in potential, for example a voltmeter, 31 shown schematically like the current generator.

The measuring device further comprises a sensor configured to record a mechanical signal indicative of a structure, shape or morphology of the digestive tract in which the measuring device is implanted. According to the illustrated example, the sensor is an accelerometer 100 accommodated in the housing. The accelerometer 100 is placed in the housing in a substantially central position, in the vicinity of the current generator 30.

The measuring device 1 also comprises a calculating module 5 capable of receiving a difference in electrical potential measured between two electrodes of the set 3 of electrodes, so as to calculate a bio-impedance value of the digestive tract and signal values measured by the sensor, here the accelerometer 100.

In a first embodiment, illustrated in FIG. 1, the set 3 of electrodes comprises at least two electrodes 6, 7 arranged in the volume of the housing and which participate, by their respective electrical connection to the current generator 30, in the formation of a closed flow loop when the measuring device 1 is positioned in a conductive medium, and more particularly in the conductive tissues of the user, as described in FIG. 2.

The housing 2 is a closed structure having an outer surface 8. The housing 2 extends its largest dimension along a longitudinal axis X. For example, the housing 2, measured along the longitudinal axis X, measures between 2 and 4 cm.

The housing 2 comprises a first longitudinal end 9 and a second longitudinal end 10 opposite the first longitudinal end 9. In the embodiment of FIG. 1, a first electrode 6 of the set 3 of electrodes is arranged at the first longitudinal end 9 of the housing 2 and a second electrode 7 of the set 3 of electrodes is arranged at the second longitudinal end 10 of the housing 2. In this embodiment, the first electrode 6 and the second electrode 7 are connected to the outer surface 8 of the housing 2 and respectively cover the first longitudinal end 9 and the second longitudinal end 10. The electrodes are shown in gray to facilitate their detection by the reader.

The housing 2 is made of an insulating material, so that the housing 2 in itself is not electrically conductive.

Each electrode of the set 3 of electrodes has an interface surface intended to be in contact with a gastric or intestinal tissue of the user. Each electrode of the set 3 of electrodes is able to function as a current injecting electrode and/or as a measuring electrode making it possible to measure a difference in electrical potential from one electrode to another while the current having to flow from one electrode to the other to close the flow loop goes through the gastric or intestinal tissue. The same electrode of the set 3 of electrodes can simultaneously have these two functionalities, transmitting and receiving, in the embodiments illustrated here. However, these functionalities can also alternate without departing from the scope of the invention.

The housing 2 is made of a biocompatible material. It is of parallelepipedal shape and it comprises rolled edges 11 so as to preserve the tissues which surround it once it is implanted. The housing 2 can take any form compatible with its use. Advantageously, the housing 2 is oblong in shape for a reduced impact on the surrounding tissues and to facilitate its positioning.

The calculating module 5 is shown here as being externalized with respect to the housing 2. It comprises means of communication so as to receive the measured electrical potential. For example, the calculating module 5 comprises a receiver. The set 3 of electrodes is then connected to a transmitter making it possible to transmit the information measured by the means for measuring the difference in electrical potential to the calculating module 5, as illustrated by waves 12. The transmitter here is comprised in the housing 2, and connected to the set 3 of electrodes. In a variant that is not illustrated, provision could be made for the calculating module to be integrated into the housing.

FIG. 2 shows the device 1 for measuring congestion of the digestive tract implemented during a method for measuring congestion of the digestive tract, the measuring device 1 here being a gastric or intestinal device implanted in a submucosa. This implantation is not limiting, and it is understood that the device according to the invention can be implanted in any other tissue of the digestive tract.

The set 3 of electrodes is configured to generate at least the flow loop 13 of the electric current when the current generator 30 is activated. This flow loop 13 then circulates at least from one electrode to another through a tissue of the gastrointestinal tract 14 of the user as shown in FIG. 2. In this embodiment, the housing 2 and the set 3 of electrodes are arranged in such a tissue 14. In particular, the housing 2 and the set 3 of electrodes are integrated into a submucosal tissue 15 of the tissue 14 of the user. Thus, the flow loop 13 circulates at least through the submucosal tissue 15 of the tissue 14 of the user.

In one embodiment, the flow loop 13 passes through the submucosal tissue 15 and a mucosa 16 of the tissue 14, the mucosa 16 separating the submucosal tissue 15 from a cavity 17 delimited by the tissue 14 and being included in the tissue 14. Depending on the position of the set 3 of electrodes, part of the flow loop 13 may be caused to flow out of the tissue 14, for example into the cavity 17 delimited by the tissue of the gastrointestinal tract 14.

It should be understood that the housing 2 and the set 3 of electrodes could be arranged differently without departing from the scope of the invention, since the position of the set 3 of electrodes allows the formation of a closed flow loop 13 passing through at least the tissue of the gastrointestinal tract 14.

The method of measuring congestion of the digestive tract, implemented by the measuring device 1, comprises at least one step of measuring the difference in electrical potential and a calculation step. It also comprises a mechanical measurement of a structural or morphological characteristic of the gastric wall, and in particular a measurement of waves following a cardiac shock by means of an accelerometer 100.

During the step of measuring the difference in electrical potential, at least one electrode, here the first electrode 6, of the set 3 of electrodes transmits the current coming from the generator 30 into the mucosa or the submucosa, this current being brought through the tissue of the gastrointestinal tract 14 in the direction of another electrode, here the second electrode 7, of the set of electrodes. The current takes the shortest route to close the flow loop 13.

A voltmeter, forming means for measuring the difference in potential 31, is connected across the terminals of the current generator to determine a measurement of the difference in electrical potential from one electrode to the other, this measurement of the difference in electrical potential being the reflection of the ease of current circulation through the gastric tissues and therefore the reflection of the presence of liquid in these tissues, as a state of bio-impedance of the digestive tract can be deduced therefrom.

The received information on the difference in electrical potential is transmitted to the calculating module 5, here by means of the transmitter which is internalized in the housing in this embodiment.

The calculation step is implemented by the externalized calculating module 5, not shown here but visible in FIG. 1. By measuring a difference in electrical potential, the calculating module 5 calculates a bio-impedance value of the digestive tract. The result is then informative as to the state of bio-impedance of the user's digestive tract, and for example makes it possible to send the user or appropriate medical personnel information on the state of the user's health, and for example on a possible state of cardiac decompensation of the user. It will be noted that the use of the measurement of a bio-impedance of the user's digestive tract is not limited to providing information on the state of cardiac decompensation of the user, and that it can make it possible to characterize other information relating to the user's health condition.

The mechanical measurement, and in particular the accelerometric measurement, provided additionally and simultaneously with the bio-impedance measurement, is carried out in response to a cardiac shock occurring during the measurement period. The accelerometer is able to quantify the wave caused by the cardiac shock as it is transmitted through the gastric wall. Variations over time of the mechanical signal thus acquired are in particular due to a structural modification of the gastric wall, which does not transmit the waves in the same way depending on its configuration.

The values collected by the accelerometer are sent to the calculating module to extract the accelerometric signature of the gastric wall in response to the cardiac shock.

The previously mentioned calculation step, implemented by the calculating module, then takes account of the acquired mechanical signals. Advantageously, the same calculating module is used to recover and process the electrical and mechanical data acquired by the sensors and electrodes.

Owing to the measurement of at least one characteristic of the accelerometric signal, the calculating module 5 calculates a value representative of a structural or morphological characteristic of the digestive tract, in the same area as that on which the bio-impedance measurement was done. The combination of these two values, and in particular the combination of the comparisons of these values with a threshold value associated with them and stored in a memory of the calculating module, is then informative as to the presence of liquid in the user's digestive tract and makes it possible, for example, to send the user or appropriate medical personnel information on the user's health condition, and for example on a possible state of cardiac decompensation of the user.

FIG. 3 illustrates an embodiment in which the measuring device 1 differs from that described in FIG. 1 as follows. The measuring device 1 comprises, in its set 3 of electrodes, an electrode called the third electrode 18, which is offset relative to the housing 2. The first electrode, the second electrode and the third electrode are electrically connected to the current generator 30, independently of one another, so as to participate in the transmission of a current carrying loop which thus travels through a larger overall distance through the tissue 14 between the first electrode 6 and the third electrode 18. The measurement is not located on the longitudinal dimension of the housing 2, but over a distance going from the first electrode 6 to the third electrode 18. More particularly, and as can be seen in FIG. 4, the overall distance between the first electrode and the third electrode is traveled by a first flow loop 13 extending between the first electrode and the second electrode, and by a second flow loop 13 extending between the second electrode and the third electrode at a distance from the housing. It will be understood that studying the electrical potentials for each of the loops makes it possible to globally consider the conductivity of the tissues over a distance going from the first electrode to the third electrode.

In this embodiment, the first electrode 6 and the second electrode 7 are connected to the outer surface 8 of the housing 2. Each electrode of the set 3 of electrodes has an interface surface 19, oriented in the same direction and intended to be in contact with the tissue 14. The normals of the interface surfaces 19 are parallel to each other and, moreover, the interface surfaces 19 have the same orientation with respect to the tissue 14, for example with respect to the submucosal tissue 15. The interface surfaces 19 take the form of pellets having any shape as long as the relative positioning of the interface surfaces 19 is respected.

The third electrode 18 here is identical to the first electrode 6 and to the second electrode 7, except that it is offset from the housing 2. An offset electrode support 20, external to the housing 2, extends from the second longitudinal end of the housing so as to support at least the third electrode 18, at a distance from this housing. The third electrode 18 is electrically connected to the current generator 30, independently of the other electrodes. To this end, the offset electrode support 20 comprises at least one electrically conductive cord and an insulating peripheral sheath 21 extending from the housing to the third electrode 18.

It should be noted that the measuring device again comprises an accelerometer 100, which this time is shifted close to the second electrode 7, in order to take a position as centered as possible over the distance between the two electrodes furthest from one another. In accordance with the first embodiment previously described, it is sought here to center the area of the gastric tract in which the measurement of mechanical signals is carried out on the area of this gastric tract in which the bio-impedance measurement is carried out.

FIG. 4 in particular shows the flow loop 13 generated by the second electrode 7 and the third electrode 18. The figure also illustrates the flow of current toward the third electrode, along the electrically conductive cord in the offset electrode support 20 as signified in dotted lines. For the other elements illustrated in FIG. 4, reference may be made, mutatis mutandis, to the description given for FIG. 2. Reference may thus be made to FIG. 2 for the implementation and understanding of the invention according to the embodiment described in FIG. 4.

The electric current is transmitted from the generator at three points of the set 3 of electrodes, that is to say, at each of the electrodes, such that they are electrically insulated from one another. As illustrated, a greater inter-electrode distance separates the second electrode 7 and the third electrode 18 compared to the first electrode 6 and the second electrode 7. The flow loop 13 generated between the second electrode 7 and the third electrode 18 is therefore larger than that generated between the first electrode 6 and the second electrode 7, the current having more distance to travel to connect the second electrode 7 and the third electrode 18. It will be understood that step by step measurement as illustrated makes it possible to prevent the flow loop 13 from deviating too far from the set 3 of electrodes and passing through the cavity 17. The electrodes are thus positioned and arranged relative to each other so as to be able to form flow loops 13 avoiding the cavity 17, inside which the gastric liquid could form an electrical shunt.

Such a measuring device 1 generates two local measurements of the difference in electrical potential, carried out between two immediately adjacent electrodes by the means for measuring a difference in electrical potential. The measurement of the difference in electrical potential used to calculate the bio-impedance value of the digestive tract corresponds to the sum of these two local measurements of the difference in electrical potential, since the current injected locally during the measurements of difference in potential is the same from one local measurement to another. Alternatively, provision may be made to determine local bio-impedance values, which are calculated respectively from local measurements of the difference in electrical potential. These local bio-impedance values can then be added to determine a digestive tract bio-impedance value corresponding to a measurement over a more global, and therefore more reliable, extent.

FIG. 5 illustrates a third embodiment in which the offset electrode support 20 serves as a support, between the second longitudinal end of the housing and the third electrode 18 arranged at the free end of the support 20, for one or more intermediate electrodes 23. With the exception of what concerns the series of intermediate electrodes, the description of the measuring device 1 according to the second embodiment as presented in FIG. 3 applies mutatis mutandis to the description of FIG. 5, and reference may be made to it for the implementation and understanding of the invention.

In this embodiment and in those which follow, in which at least two electrodes are offset outside the housing, with at least the third electrode 18 arranged at the end of the support 20 and at least one intermediate electrode arranged on this support between the third electrode and the housing, provision may be made, as shown in FIGS. 9 to 12 for example, for all the electrodes to be offset and for all the electrodes to thus extend outside the housing.

In this case, the series of intermediate electrodes comprises four intermediate electrodes 23. Like the third electrode 18, the intermediate electrodes 23 are connected to the offset electrode support 20. The offset electrode support 20 is a direct support for the electrodes of the series of intermediate electrodes, just like for the third electrode 18.

Each intermediate electrode 23 is here configured to both transmit an electric current and at the same time recover an electric signal to allow the measurement of an electrical potential. In this, all the electrodes of the set 3 of electrodes, including the intermediate electrodes 23, can be similar.

Here, each intermediate electrode 23 is separated from the series 22 of intermediate electrodes by an equal distance. For the whole of the measuring device 1, each electrode of the set 3 of electrodes is separated from the immediately adjacent electrode by this same distance.

Each intermediate electrode 23 is electrically connected, independently of the other electrodes, to the electric generator 30 and/or to the means for measuring a difference in electrical potential. As described above, these electrical connections are made in particular by conductive cords independently of each other and respectively connecting an electrode to the generator.

FIG. 6 shows the measuring device 1 illustrated in FIG. 5, implanted in the submucosal tissue 15. For the other elements illustrated in FIG. 6, reference may be made, mutatis mutandis, to the description given for FIG. 2. Reference may thus be made to FIG. 2 for the implementation and understanding of the invention according to the embodiment described in FIG. 6.

In this embodiment, the distance, here equal, which separates the immediately adjacent electrodes of the set 3 of electrodes allows local flow loops 13 to form, each closed for two adjacent electrodes of the set 3 of electrodes, step by step. The electrodes of the set 3 of electrodes are able to transmit in several directions so that the local flow loops 13 pass through one or the other of the adjacent tissues of the set 3 of electrodes.

Thus, in the method of measuring congestion of the digestive tract according to the invention, the bio-impedance measurement can be determined via a measurement of the difference in global electrical potential calculated by adding up a plurality of local measurements of difference in electrical potential, since the current injected locally during the measurements of differences in potential is the same from one local measurement to another, each local measurement of the difference in electrical potential resulting from a measurement done across the terminals of an electrode pair of the measuring device 1. Alternatively, provision may be made to determine local bio-impedance values, which are calculated respectively from local measurements of the difference in electrical potential. These local bio-impedance values can then be added to determine a digestive tract bio-impedance value corresponding to a measurement over a more global, and therefore more reliable, extent.

FIG. 7 and FIG. 8 and illustrate embodiments where the third electrode 18 and the intermediate electrodes 23 of the series of intermediate electrodes each comprise their interface surface 19 with the tissue of the gastrointestinal tract 14 of the user's body such that the interface surfaces 19 of the third electrode 18 and of the intermediate electrodes 23 are oriented in the same direction and in the same sense. The direction and the sense are assessed with respect to the normal to the extension plane of each interface surface 19, as previously described. In particular, the interface surfaces 19 of the first electrode 6 and the second electrode 7 are also oriented in this same direction. When positioning the measuring device 1, it is possible to aim for each interface surface 19 of the set 3 of electrodes to be positioned so as to be directed at the heart of the tissue of the gastrointestinal tract 14, in a specific manner. This configuration allows the local flow loops 13 to travel in their entirety through the tissue of the gastrointestinal tract 14, here the submucosal tissue 15. In the set 3 of electrodes, each interface surface 19 can be opposite an electrically insulating surface making it possible to specifically orient the current injected into the tissue and thus prevent the flow loop 13 from forming on the side of the device opposite the electrodes. For example, the electrically insulating surface is covered with silicone.

In the example of FIG. 7, the series of intermediate electrodes 23 and the third electrode 18 are implanted within the submucosal tissue 15. Each interface surface 19 of the set of electrodes 3 is positioned so that it faces the heart of the submucosal tissue 15, and not the side of the mucosa 16.

In the example of FIG. 8, each interface surface 19 of the set 3 of electrodes is arranged on the surface of the gastric tissue 14 opposite the stomach cavity 17. It will be understood that the illustration of FIG. 8 is schematic and that an additional tissue layer could be placed between the submucosal tissue 15 and the electrode interface surfaces since, in accordance with what has just been described, the electrodes are arranged opposite the stomach cavity 17 with respect to the tissue of the digestive tract. In other words, the set 3 of electrodes could be placed in contact with any other gastric tissue 14 without departing from the scope of the invention, since each interface surface 19 of the set 3 of electrodes is positioned so as to be directed toward the heart of said gastric tissue 14.

To allow this configuration described in FIG. 8 and to keep the set 3 of electrodes in position, the measuring device 1 comprises at least one device 24 for attaching to the tissue of the gastrointestinal tract 14. Here, each end of the housing 2, namely the first longitudinal end 9 and the second longitudinal end 10, comprises a device 24 for attaching to the tissue 14, which makes it possible to maintain the position of the first electrode 6 and of the second electrode 7. One or more attachment devices can be distributed on the offset electrode support 20. Advantageously, an attachment device 24 is located at least in the vicinity of the third electrode 18 so as to hold the third electrode 18, which is the electrode furthest from the housing 2.

It will be understood that the advantage of such attachment devices is just as strong for the devices of the other embodiments, and that it may be provided according to the invention to equip the housings or the electrically conductive cords with such an attachment device.

FIG. 9 illustrates an embodiment of the invention, in which the third electrode 18 and the intermediate electrodes 23 are laterally offset with respect to the direction of elongation of the offset electrode support 20. The third electrode 18 and the intermediate electrodes 23 are each connected to the offset electrode support 20 by a current conducting branch 25. The insulating peripheral sheath 21 is configured to also cover each of the current conducting branches 25, or else insulating sheaths specific to each branch are connected to the insulating peripheral sheath 21. The current is thus able to flow from the current generator 30, via the offset electrode support 20, to each of the laterally offset electrodes, so that this position does not prevent all the electrodes from being connected to the electric generator independently of each other.

As mentioned above, this embodiment is also particular in that the first electrode and the second electrode previously present in the housing here have been removed. In this way, the set of electrodes is offset and all the electrodes respectively supplied by the generator are placed at a distance from the housing. It will be understood that the previously described embodiments in which at least two electrodes extend outside the housing could also be arranged in this way, with no electrode integrated into the housing.

FIG. 10 shows the device 1 for measuring congestion of the digestive tract in situ, implanted in the tissue of the gastrointestinal tract 14 of the user. As shown in FIG. 8, each electrode of the set 3 of electrodes generates local flow loops 13 within the submucosal tissue 15. The local flow loops 13 are laterally offset with respect to what has been shown in FIG. 8, so as to be remote from the offset electrode support 20 and, therefore, from the mucosa 16 located in the vicinity of this offset electrode support 20. The local flow loops 13 then pass through healthy tissue or tissue which is less impacted by fibrous degeneration which may result from the implantation of the offset electrode support 20. Thus, the local measurements of differences in electrical potential are independent of artifacts that may occur with fibrous degeneration of tissue in the gastrointestinal tract 14.

FIG. 11 and FIG. 12 illustrate the measuring device 1 in which the current conducting branch 25 is deployable in the tissue of the gastrointestinal tract 14. The description given in FIG. 9 applies mutatis mutandis to the description of FIGS. 11 and 12, and reference may be made to the description of FIG. 9 in order to implement and understand the invention.

FIGS. 11 and 12 show an embodiment of the deployability of the current conducting branch 25. The deployable current conducting branch 25 is used, for example, during the implantation of the offset electrode support 20 within the tissue of the gastrointestinal tract 14. The deployability of the current conducting branch 25 then allows the electrodes to be moved away from the set 3 of electrodes without tearing the gastric tissue, and therefore with a lower impact on the tissue of the gastrointestinal tract 14. The deployable current conducting branch 25 may also be useful for orienting the electrodes in the tissue of the gastrointestinal tract 14 or on the outer surface 8 thereof.

In this way, the drawback of implanting a device with branches already deployed is avoided, which would have the effect of tearing fibers on insertion and of again creating a reformation of fibers around the electrodes.

When installing the device 1 for measuring congestion of the digestive tract in the user, the current conducting branches 25 are liable to catch in the tissues during installation of the measuring device 1. It is thus possible to cover the offset electrode support 20 with a protective case 26 as shown in FIG. 11. This protective case 26 will be removed once the measuring device 1 has been positioned. The protective case 26 is removed as shown in FIG. 12.

As shown in FIG. 11, when the offset electrode support 20 is covered with the protective case 26, the current conducting branches 25 are preferably in the folded or retracted position along the offset electrode support 20 to occupy a smaller volume and prevent them from being damaged.

As shown in FIG. 12, when the offset electrode support 20 is uncovered and the protective case 26 is removed, the current conducting branches 25 move to the extended position as shown by the solid arrow 28. To do this, a deployment mechanism, not shown here, is actuated for example automatically when the protective case 26 no longer constrains the deployment of the current conducting branch 25.

The protective case 26 is removed once the offset electrode support 20 has been positioned for implementation. Preferably, the protective case 26 is removed in a direction opposite the direction of insertion of the housing 2 as illustrated by an arrow 27, thus using a passage made in the user when the housing 2 is inserted.

FIG. 13, FIG. 14 and FIG. 15 illustrate appropriate electrical connection diagrams for the correct operation of the measuring device according to the invention.

FIG. 13 illustrates a control unit comprising a plurality of submodules 300, 301, 302 respectively forming a control chip integrating the current generator and the means for measuring the difference in electrical potential. Each submodule 300, 301, 302 is electrically connected to two electrodes, with at least one of the electrodes that it connects also being connected to another submodule 300, 301, 302. By way of example, a first submodule 301 is connected to the first electrode 6 and to the second electrode 7 and a second submodule 302 is connected to this same second electrode 7 and an intermediate electrode 23. When current is generated at the first submodule 301, a switch 32 ensures that the second electrode 7 is connected to this first submodule 301 and the measurement of the difference in potential is done at the first submodule 301 between the first electrode 6 and the second electrode 7. When current is then generated at the second submodule 302, a switch 33 ensures that the second electrode 7 is connected this time to this second submodule 302 and that the adjacent electrode, namely here the intermediate electrode 23, is also connected to the second submodule 302: the measurement of the difference in potential is then done at the second submodule 302 between the second electrode 7 and the intermediate electrode 23.

FIG. 14 and FIG. 15 illustrate measurement variants with two or four electrodes, in particular in the context of a plurality of local measurements making it possible to obtain an overall measurement of the difference in potential.

In FIG. 14, for example, a control of the switches at the current generator 30 has been illustrated which makes it possible to form flow loops step by step including two immediately adjacent electrodes. Here we are talking about local measurements with two electrodes. More particularly, the connection of these electrodes is illustrated in FIG. 16. For a series comprising N electrodes 23, 18, from the housing to the free end of the electrode support on which the third electrode is placed, it is understood that when a switch is controlled to supply current to an electrode n, a switch is then driven to electrically connect the directly adjacent electrode n+1 to the same current generator so that the associated voltmeter retrieves information on the difference in electrical potential, then this same switch is driven this time to connect the electrode n+1 and the electrode n+2 directly adjacent to the same current generator so that the associated voltmeter recovers another information item on the difference in electrical potential, and so on, step by step, to recover all the information on the differences in electrical potential.

FIG. 15 illustrates a variant of the device according to the invention in which a local measurement with four electrodes is implemented, with sets of four electrodes which can be formed step by step by the appropriate control of the switches. More particularly, the connection of these sets of electrodes is illustrated in FIG. 17. The switches are controlled so that the electrodes are energized four by four, in sets of two electrodes. The global measurement is again done by taking several local measurements into account.

As mentioned previously, FIG. 16 illustrates an assembly allowing a measurement with two electrodes, the means for measuring a difference in potential 31 being connected across the terminals of the voltage generator 30 to which each of the electrodes 23 are respectively connected, embedded here in the submucosal tissue 15. And FIG. 17 illustrates a different setup from that shown in FIG. 16, in that it allows a four-electrode measurement, with a pair of electrodes connected to the generator for the formation of the flow loop in the submucosal tissue 15, and a pair of electrodes connected directly to the means 31 for measuring a difference in potential. A four-electrode measurement minimizes the contact impedance due to the electrodes.

The various embodiments previously described and illustrated can be implemented with one or the other of these assemblies.

It will be understood from reading the foregoing that the present invention proposes a device for measuring congestion of the digestive tract configured to make the detection of pulmonary edema indicative of cardiac decompensation reliable. This measuring device, intended in particular to be implanted in or against the gastric tissues of a user, facilitates the regular monitoring of at-risk users. The efficiency of the bio-impedance measurement of the digestive tract is increased in the various embodiments of the invention, owing to the tissue specificity and the positioning stability of the electrodes used. Furthermore, the combination of this bio-impedance measurement with a separate additional mechanical measurement, capable of highlighting indications of changes in the morphology or structure of the gastric wall, makes it possible to make the analysis relating to the excessive water content of the gastric wall more reliable.

The invention cannot, however, be limited to the means and configurations described and illustrated here, and it also extends to any equivalent means or configuration and to any technical combination using such means. In particular, the shape of the measuring device can be modified without harming the invention, insofar as the measuring device, in fine, fulfills the same functionalities as those described in this document.

1. Device for measuring (1) congestion of the digestive tract of a user, characterized in that it comprises at least one housing (2), a current generator (30) and a means (31) for measuring a difference in potential, said generator and means being accommodated in said housing, and a set of electrodes (3) comprising at least two electrodes connected electrically, independently of one another, to the current generator (30) and/or to the means (31) for measuring a difference in potential across the terminals of the electrodes, each electrode of the set (3) of electrodes being configured to transmit an electric current and/or to allow the measurement of a difference in electrical potential, and characterized in that the set (3) of electrodes is configured to generate at least one electric current flow loop (13) flowing at least through a tissue of the gastrointestinal tract (14) of the user and to allow a difference in electrical potential in relation to the tissue of the gastrointestinal tract (14) to be measured, the measuring device (1) further comprising a calculating module (5) configured to receive the measured difference in electrical potential and to calculate a value of the bio-impedance of the digestive tract according to this measurement in relation to the tissue of the gastrointestinal tract (14).

2. Measuring device according to claim 1, characterized in that it comprises means (100) allowing a mechanical measurement of a structural or morphological characteristic of the gastric wall.

3. Measuring device according to claim 1, characterized in that the means allowing a mechanical measurement of a structural or morphological characteristic of the gastric wall comprise an accelerometer (100).

4. Measuring device according to claim 2, characterized in that the means (100) allowing a mechanical measurement of a structural or morphological characteristic of the gastric wall are integrated into the housing (2).

5. Measuring device (1) according to claim 1, wherein the set (3) of electrodes comprises two electrodes, namely a first electrode (6) arranged at a first longitudinal end (9) of the housing (2) and a second electrode (7) arranged at an opposite second longitudinal end (10) of the housing (2).

6. Measuring device (1) according to claim 1, wherein the set (3) of electrodes comprises a plurality of electrodes among which at least one electrode (18, 23) is offset outside the housing (2), and in which an offset electrode support (20) external to the housing (2) is configured to connect the current generator (30) and/or the means for measuring a difference in potential (31) to said offset electrode (18, 23), the offset electrode support (20) comprising an insulating peripheral sheath (21) extending between the housing (2) and said offset electrode (18).

7. Measuring device (1) according to claim 6, wherein all the electrodes of the set of electrodes (3) are offset outside the housing, the insulating peripheral sheath (21) accommodating, in a manner electrically insulated from one another, a plurality of electrically conductive cords configured to connect the offset electrodes to the current generator (30) and/or to the means for measuring a difference in potential (31), independently of each other.

8. Measuring device (1) according to claim 6, wherein the set of electrodes comprises at least one intermediate electrode (23) extending outside the housing between the housing (2) and said offset electrode (18) forming the electrode furthest from the housing, at least one intermediate electrode (23) being arranged at the end of a current conducting branch (25) and insulated from the tissue of the gastrointestinal tract by an insulating sheath, said branch (25) being deployable relative to the support (20) provided with the peripheral insulating sheath (21).

9. Measuring device (1) according to claim 6, wherein the set of electrodes comprises a plurality of electrodes electrically connected to the current generator (30) and/or to the means for measuring a difference in potential (31), the measuring device comprising a set of switches (32) arranged on the electrical connections independently of each electrode with the current generator (30) and/or the means for measuring a difference in potential (31), the calculating module (5) being configured to drive the switch to determine which electrodes in the set are implemented to transmit and measure the current through the tissue of the gastrointestinal tract.

10. Measuring device (1) according to claim 1, wherein each electrode is connected both to the current generator (30) for carrying out the transmission into the tissue of the gastrointestinal tract of an electric current and to the means for measuring the difference in potential (31).

11. Measuring device (1) according to claim 1, wherein at least one pair of electrodes is connected across the terminals of the current generator (30) to participate in the transmission of a current flow loop in the tissue of the gastrointestinal tract, and at least one pair of electrodes is connected across the terminals of the means for measuring a difference in potential (31).

12. Measuring device (1) according to claim 1, comprising at least one attachment device (24) for attaching to the tissue of the gastrointestinal tract (14), arranged at least at one end of the housing (2) and/or on the offset electrode support (20) and/or on the current conducting branch (25).

13. Method for measuring congestion of the digestive tract, implementing the measuring device (1) for measuring congestion of the digestive tract according to one of the preceding claims, the method comprising a step of measuring the difference in electrical potential and a calculating step, the calculating step being implemented by a calculating module (5) capable of receiving at least one measurement of the difference in electrical potential and of calculating a bio-impedance value of the digestive tract from the measurement of the difference in electrical potential.

14. Method for measuring congestion of the digestive tract according to the preceding claim, wherein the measurement of the difference in electrical potential corresponds to the sum of a plurality of local measurements of the difference in electrical potential, each local measurement of the difference in electrical potential resulting from an electrical connection to at least one current generator (30) of an electrode or of a pair of immediately adjacent electrodes of the set (3) of electrodes of the measuring device (1).
